(19) United States
(12) Patent Application Publication
(10) Pub. No.: US 2021/0296005 A1
(43) Pub. Date: Sep. 23, 2021

(54) NON-TRANSITORY COMPUTER-READABLE STORAGE MEDIUM FOR STORING INFORMATION PRESENTATION PROGRAM, INFORMATION PRESENTATION METHOD, AND INFORMATION PRESENTATION DEVICE

(71) Applicant: FUJITSU LIMITED, Kawasaki-shi (JP)

(72) Inventors: Hidekazu TAKAHASHI, Kawasaki (JP); YUTAKA MITSUISHI, Kawasaki (JP)

(73) Assignee: FUJITSU LIMITED, Kawasaki-shi (JP)

(21) Appl. No.: 17/175,738

(22) Filed: Feb. 15, 2021

(30) Foreign Application Priority Data
Mar. 18, 2020 (JP) 2020-048424

(51) Int. Cl. G16H 50/70; G16H 70/60; G16H 70/20; G16H 50/20; G16H 15/00; G06F 16/903; G06F 16/9038

(52) U.S. Cl. CPC G16H 50/70; G16H 70/60; G16H 70/20; A61B 5/7267; G16H 15/00; G06F 16/90344; G06F 16/9038; G16H 50/20

(57) ABSTRACT

A method includes: in response to a target disease name and an input practice information on the target disease, detecting a similar disease name from a list of disease names, the similar disease name having a character string similar to the target disease name, the input practice information indicating an examination result when the target disease name is determined; obtaining a determination result by inputting input information to a task, the input information including the similar disease name and the input practice information, the task being configured to determine whether the similar disease name corresponds to any one of a plurality of rules, each of the plurality of rules defining a relationship between a disease name and a practice information on the disease name; and in response to the determination result indicating that the similar disease name corresponds to any of the plurality of rules, displaying the similar disease name.

### CROSS-REFERENCE TO RELATED APPLICATION

This application is based upon and claims the benefit of priority of the prior Japanese Patent Application No. 2020-48424, filed on Mar. 18, 2020, the entire contents of which are incorporated herein by reference.

### FIELD

The embodiment discussed herein is related to a non-transitory computer-readable storage medium storing an information presentation program, an information presentation method, and an information presentation device.

### BACKGROUND

Conventionally, in a medical care field, a diagnosis support processing device using information processing is known. This diagnosis support processing device refers to a diagnosis rule base in which a disease name and a symptom are associated with each other on the basis of a symptom profile, and acquires information (score) indicating a relationship between a medical proposition information to be diagnosed and the disease name in the diagnosis rule base. Then, this diagnosis support processing device determines the disease name corresponding to the propositional information on the basis of the information indicating the relationship. The propositional information includes medical records, images illustrating symptoms of disease and the like of electronic medical records.

Furthermore, a disease diagnosis support device that enables disease diagnosis support by reflecting vital signs, daily physical condition, and medical history in consideration of an individual difference of a subject is known. This disease diagnosis support device determines whether a vital value of the subject on the measured day is abnormal based on vital information of the subject. This disease diagnosis support device determines whether the subject has a specific disease in a case where the vital information and health observation information obtained from the subject satisfy a predetermined condition.

A diagnostic support device is known that supports an operator such as a doctor to determine an appropriate disease name by providing information for verifying a disease name based on findings to the operator. This diagnosis support device uses a database in which a disease name and findings corresponding to the disease name are associated with each other, receives a focused disease name, and searches the database for findings corresponding to the focused disease name, another disease name (similar disease name) commonly corresponding to the findings, and findings corresponding to the similar disease name and displays the same on a screen.

Furthermore, a database in which a disease name term is standardized for each disease concept is known. Furthermore, a clinical medicine ontology that describes a conceptual definition of a disease on a computer in order to enable semantic processing of a disease name term is known.

Examples of the related art include Japanese Laid-open Patent Publication No. 2017-167738, Japanese Laid-open Patent Publication No. 2017-131495, Japanese Laid-open Patent Publication No. 2009-069893, and OHE Kazuhiko, “Standardization of Disease Names and Development of an Advanced Clinical Ontology”, Journal of Information Processing and Management, vol. 52, no. 12, pp. 701-709, 2009, Published on Mar. 1, 2010.

### SUMMARY

According to an aspect of the embodiments, a method implemented by a computer includes: in response to inputting of a target disease name and an input practice information with respect to the target disease, detecting a similar disease name from a list of disease names, the similar disease name being a disease name having a character string similar to a character string representing the target disease name by a predetermined criterion, the input practice information indicating an examination result when the target disease name is determined in a practice; obtaining a determination result by inputting input information to an inference task, the input information including the detected similar disease name and the input practice information, the inference task being configured to determine whether the similar disease name corresponds to any one of a plurality of practice rules, each of the plurality of practice rules defining a relationship between a corresponding disease name and a practice information with respect to the corresponding disease name; and in response to the determination result indicating that the similar disease name corresponds to any one of the plurality of practice rules, displaying the similar disease name.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is an illustration diagram for illustrating an outline of an information presentation device of this embodiment;

FIG. 2 is a schematic block diagram of an information presentation system according to the embodiment;

FIG. 3 is a view illustrating an example of disease name ontology;

FIG. 4 is a view illustrating an example of disease name ontology;

FIG. 5 is a view illustrating an example of a practice rule;

FIG. 6 is an illustration diagram for illustrating determination of whether it corresponds to the practice rule;

FIG. 7 is an illustration diagram for illustrating determination of correspondence of the practice rule based on an inclusion relationship;

FIG. 8 is a view illustrating an example of information stored in a relationship table storage unit;

FIG. 9 is a view illustrating an example of information stored in the relationship table storage unit;

FIG. 10 is a view illustrating an example of information stored in the relationship table storage unit;

FIG. 11 is a block diagram illustrating a schematic configuration of a computer that serves as the information presentation device according to this embodiment;

FIG. 12 is a block diagram illustrating a schematic configuration of a computer that serves as an inference device according to this embodiment;

FIG. 13 is a view illustrating an example of a display screen presented to a user;

FIG. 14 is a flowchart illustrating an example of an information presentation routine of this embodiment;

FIG. 15 is a flowchart illustrating an example of an inference routine of this embodiment;

FIG. 16 is an illustration diagram for illustrating correspondence of the practice rule;

FIG. 17 is an illustration diagram for illustrating correspondence of the practice rule;

FIG. 18 is a view illustrating an example of a display screen presented to a user; and

FIG. 19 is a view illustrating an example of a display screen presented to a user.

### DESCRIPTION OF EMBODIMENTS

By the way, there are various designations of a disease name and they are not unified in some cases. For example, the designations of the disease name might differ for each medical worker or for each medical institution. In contrast, the disease name in a practice rule described in a medical guideline is unified. The practice rule is a rule in which a disease name, practice information indicating a pathological condition of a patient, and information regarding medical care are associated with each other. Therefore, in a case where a combination of a certain disease name and practice information of a patient corresponds to the practice rule, the information regarding medical care (for example, treatment, medication, medical knowledge and the like) may be obtained.

For example, consider a case where a certain doctor applies a disease name “diastolic failure” that he/she normally uses to the practice rule. In this case, suppose that the disease name “diastolic failure” is expressed as a disease name “heart failure” in the practice rule. At that time, a disease name dictionary in which a plurality of disease names is registered is referred to, and it is determined whether the disease name “diastolic failure” corresponds to the disease name “heart failure” in the practice rule from a relationship between the disease name “diastolic failure” and the disease name “heart failure”. The disease name dictionary describes the relationship between a plurality of disease names. For example, in the disease name dictionary, in a case where “diastolic failure” is a type of “heart failure”, the relationship that “diastolic failure” is regarded as “heart failure” and the like is described.

However, in a case where the disease name “diastolic failure” is not registered in the disease name dictionary, even though the disease name “diastolic failure” and the disease name “heart failure” correspond to each other medically, the disease names in character string are different, so that the information regarding medical care may not be appropriately derived by using the practice rule.

In contrast, for example, a method for searching the disease name dictionary for disease names similar in character string with the disease name “diastolic failure” and specifying a disease name medically similar to the disease name “diastolic failure” from the similar disease names is considered. However, in this case, there is a case where a large number of disease names that are similar in character string to the disease name “diastolic failure” but have no relation to the disease name “diastolic failure” are extracted. Therefore, there is a problem that, in a case where the disease name is not registered in the disease name dictionary, it is difficult to appropriately search for the disease name that is medically similar to the disease name.

In one aspect, an object of the disclosed technology is to appropriately search for a disease name medically similar to an input disease name even in a case where the input disease name is not registered in the disease name dictionary.

Hereinafter, an example of an embodiment of the disclosed technology is described in detail with reference to the drawings.

<Information Presentation Device of Embodiment>

A doctor decision support system based on information on a medical guideline is known. By using this decision support system, a medical worker such as a doctor may obtain information on treatment or medication recommended for a patient, for example.

A plurality of practice rules is registered in this decision support system. The practice rule is a rule that defines a relationship among a disease name, practice information, and information regarding medical care. The information regarding medical care includes, for example, treatment, medication, medical knowledge and the like to a patient according to the disease name and the practice information. When the disease name and the practice information correspond to a certain practice rule, the information regarding medical care associated with the practice rule is obtained. Therefore, for example, a medical worker such as a doctor may obtain information on treatment and medication recommended for a patient.

When using the decision support system, a disease name is desirably input. However, there are various designations of the disease name and they are not unified. Therefore, in a case where the disease name input to the decision support system (hereinafter, simply referred to as a “target disease name”) does not coincide with a disease name represented in the practice rule, the target disease name does not correspond to the practice rule even in a case where this originally corresponds to the disease name in the practice rule. In this case, the information regarding medical care for the target disease name may not be obtained.

Note that, when it is determined whether the target name corresponds to the practice rule, a disease name dictionary in which a plurality of disease names is registered is referred to, and it is determined whether the target disease name corresponds to the disease name in the practice rule. However, in a case where the target disease name is not registered in the disease name dictionary from the first, it is determined that the target disease name does not correspond to the practice rule even if this is intended to mean the disease name in the practice rule.

Therefore, in this embodiment, in a case where the target disease name for which the medical worker wants to obtain the information regarding medical care is not registered in the disease name dictionary, a disease name medically similar to the target disease name is appropriately presented.

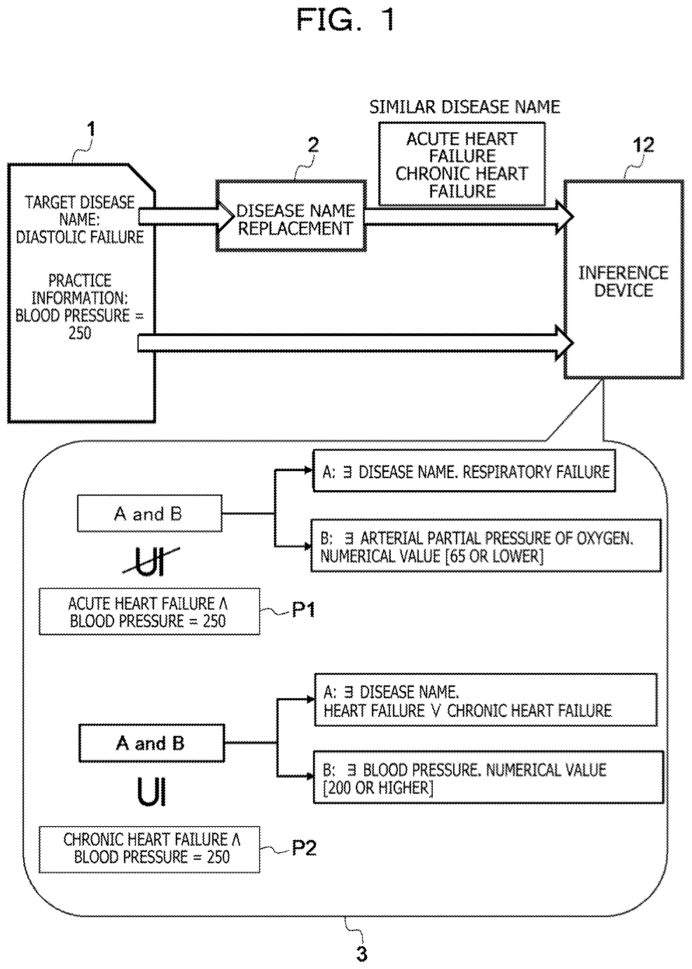

FIG. 1 is an illustration diagram for illustrating an outline of an information presentation device of this embodiment. For example, a case is considered where information 1 indicating a target disease name “diastolic failure” and practice information “blood pressure=250” indicating a pathological condition of a patient is input to the information presentation device as illustrated in FIG. 1. Here, “blood pressure=250” indicates that a measured blood pressure value of the patient is 250 [mmHg]. Note that, suppose that the target disease name “diastolic failure” is, for example, a disease name that is usually used by a doctor at a certain medical institution but is not registered in a disease name dictionary in which a plurality of disease names is registered in detail.

In a case where the target disease name “diastolic failure” is not registered in the disease name dictionary, the information presentation device of this embodiment executes processing 2 to replace the target disease name “diastolic failure” with a similar disease name that is a disease name similar to the target disease name. Suppose that the target disease name “diastolic failure” is replaced with a similar disease name “acute heart failure” and a similar disease name “chronic heart failure” by this processing 2. Note that the similar disease name similar to the target disease name is specified according to a degree of similarity in character string. The specification of the similar disease name is described later.

Next, an inference device 12 determines whether a combination of the similar disease name “acute heart failure” and the practice information “blood pressure=250” corresponds to a condition of any one of a plurality of practice rules. Furthermore, the inference device 12 determines whether a combination of the similar disease name “chronic heart failure” and the practice information “blood pressure=250” corresponds to a condition of any one of a plurality of practice rules.

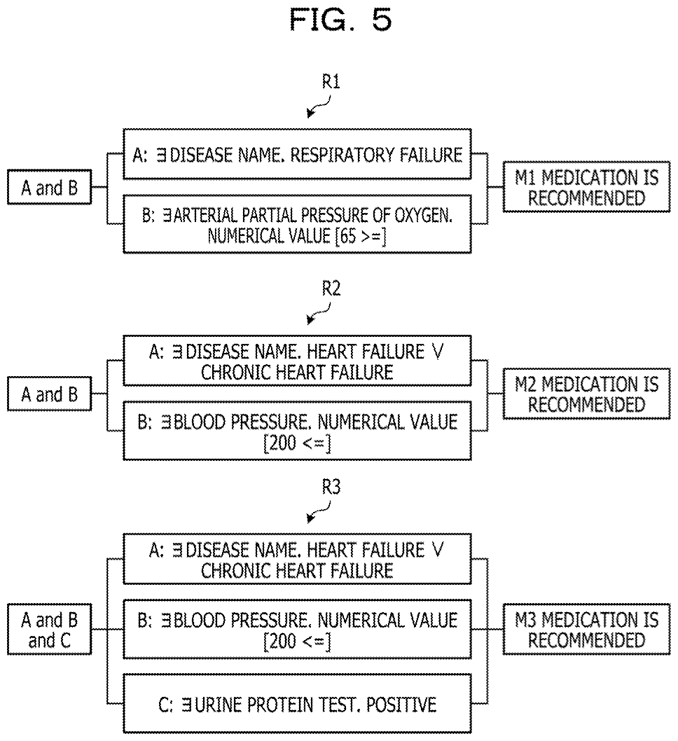

For example, as illustrated in 3 in FIG. 1, a practice rule R1 (refer to FIG. 5) includes two conditions: a condition A “ [symbol] disease name. respiratory failure” and a condition B “ [symbol] arterial partial pressure of oxygen. numerical value [65 or lower]”. Note that, here, “ [symbol] ” represents an existential quantifier. The condition A “ [symbol] disease name. respiratory failure” indicates the condition that a certain disease name corresponds to “respiratory failure”. Furthermore, the condition B “ [symbol] arterial partial pressure of oxygen. numerical value [65 or lower]” indicates that the numerical value of the arterial partial pressure of oxygen is 65 or lower. Therefore, P1 “acute heart failure ∧blood pressure=250” that is a combination of the similar disease name “acute heart failure” and the practice information “blood pressure=250” does not correspond to the condition of the practice rule R1. Note that “∧” represents a logical product, and P1 “acute heart failure∧blood pressure=250” indicates that the disease name is “acute heart failure” and the practice information is “blood pressure=250”. Therefore, since P1 does not correspond to a subset of R1, a relational expression expressed by a following expression is established between P1 “acute heart failure∧blood pressure=250” and the practice rule R1.

P1 [symbol] R1

Note that, suppose that the combination of the similar disease name “acute heart failure” and the practice information “blood pressure=250” corresponds to none of other practice rules.

In contrast, as illustrated in 3 in FIG. 1, a practice rule R2 (refer to FIG. 5) includes a condition A “ [symbol] disease name. heart failure [symbol] chronic heart failure” and a condition B “ [symbol] blood pressure. numerical value [200 or higher]”. In this case, the condition A indicates that the disease name is “heart failure” or “chronic heart failure”, and the condition B indicates that the numerical value of the blood pressure is 200 or higher. Therefore, the combination of the similar disease name “chronic heart failure” and the practice information “blood pressure=250” corresponds to the condition of the practice rule R2. Here, since P2 corresponds to a subset of R2, a relational expression expressed by a following expression is established between P2 “chronic heart failure∧blood pressure=250” and the practice rule R2.

P2 [symbol] R2

In this manner, the combination of the similar disease name “chronic heart failure” and the practice information “blood pressure=250” corresponds to the practice rule R2, whereas the combination of the similar disease name “acute heart failure” and the practice information “blood pressure=250” corresponds to none of the practice rules including the practice rule R1.

Therefore, it may be said that the similar disease name “chronic heart failure” is the disease name medically more similar to the target disease name “diastolic failure” than the similar disease name “acute heart failure”. Therefore, the information presentation device of this embodiment presents the similar disease name “chronic heart failure” that corresponds to the condition of the practice rule as a candidate disease name of the target disease name “diastolic failure”.

A medical worker such as a doctor who is a user of the information presentation device confirms “chronic heart failure” presented by the information presentation device and confirms that the target disease name “diastolic failure” input by himself/herself corresponds to “chronic heart failure”. Then, the user inputs information indicating that “chronic heart failure” is selected to the information presentation device. Then, the information presentation device associates “diastolic failure” with “chronic heart failure”. Therefore, “diastolic failure” that is not registered in the disease name dictionary and “chronic heart failure” registered in the disease name dictionary are associated with each other, and the disease name “chronic heart failure” medically similar to “diastolic failure” is obtained.

Hereinafter, the information presentation device of this embodiment is specifically described.

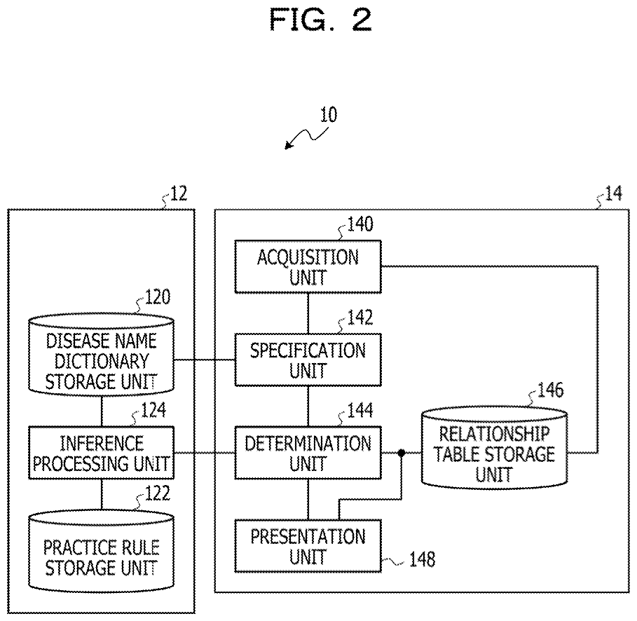

As illustrated in FIG. 2, an information presentation system 10 according to this embodiment is provided with the inference device 12 and an information presentation device 14.

The inference device 12 determines whether an input combination of a disease name and practice information corresponds to a condition of any one of a plurality of practice rules. As illustrated in FIG. 2, the inference device 12 is provided with a disease name dictionary storage unit 120, a practice rule storage unit 122, and an inference processing unit 124. The inference device 12 is an example of an inference task of the disclosed technology. The inference device 12 is a doctor decision support system based on information of a medical guideline, and is realized by, for example, clinical decision support systems (CDSS).

The disease name dictionary storage unit 120 stores the disease name dictionary. The disease name dictionary of this embodiment is, for example, ontology regarding a relationship between disease names (disease name ontology). In the disease name ontology, the relationship between a plurality of disease names is described, and a plurality of disease names is registered so as to represent an inclusion relationship between the disease names. Specifically, in the disease name ontology, a hierarchical structure according to a super-sub relationship between the disease names is defined.

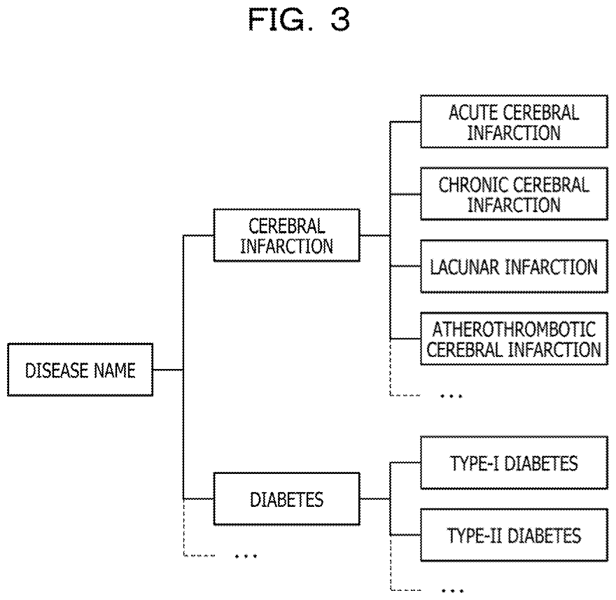

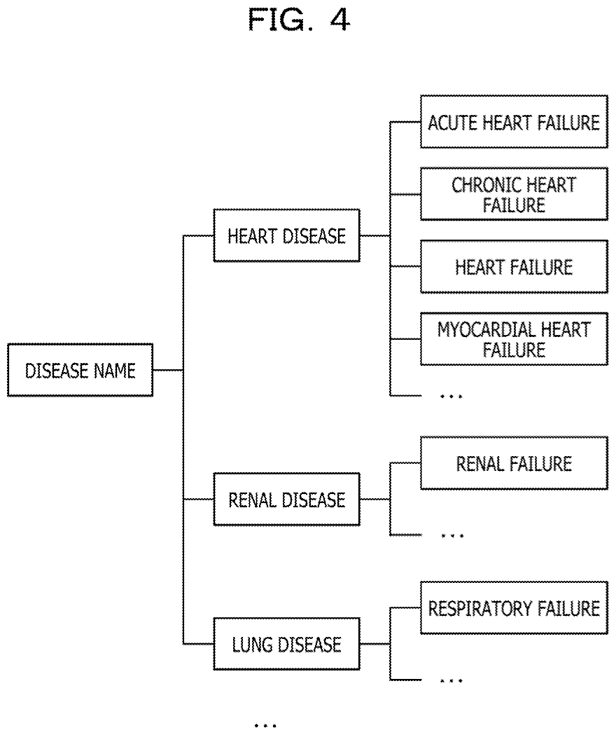

FIGS. 3 and 4 are illustration diagrams for illustrating the disease name ontology. An example illustrated in FIG. 3 illustrates an inclusion relationship of cerebral infarction and diabetes. Furthermore, an example illustrated in FIG. 4 illustrates an inclusion relationship of heart disease, renal disease, and lung disease. For example, as a subordinate concept of “cerebral infarction” illustrated in FIG. 3, “acute cerebral infarction”, “chronic cerebral infarction”, “lacunar infarction”, “atherothrombotic cerebral infarction” and the like are illustrated. Therefore, for example, the inclusion relationship is established between “cerebral infarction” and “acute cerebral infarction”.

A plurality of practice rules is registered in the practice rule storage unit 122. The practice rule defines a relationship among a disease name, practice information, and information regarding medical care.

Note that the practice information is information indicating a pathological condition of a patient. The practice information includes, for example, information such as test results, treatment contents, information on medicines being taken, symptoms, complications, medical history, and patient information (for example, age, sex and the like). Furthermore, the information regarding medical care is, for example, information such as treatment, medication, and medical knowledge. For example, a medical worker such as a doctor obtains information regarding medical care about a disease of a patient by applying a combination of a target disease name and practice information of the patient to the condition of the practice rule.

The practice rule includes, for example, a part indicating a condition that if the disease name is “cerebral infarction” and the practice information is “systolic blood pressure value (hereinafter, simply referred to as a “SBP value”)>=200” and a part indicating the information regarding medical care “antihypertensive drug M medication is recommended”.

The condition in the practice rule is expressed as follows, for example.

( [symbol] disease name. cerebral infarction) and ( [symbol] SBP value. numerical value [200<=])

“ [symbol] disease name. cerebral infarction” indicates a condition that a certain disease name corresponds to “cerebral infarction”. Furthermore, “ [symbol] SBP value. numerical value [200<=]” indicates a condition that the “SBP value” indicating the systolic blood pressure value in the practice information is 200 or higher.

For example, the practice rule storage unit 122 stores a plurality of practice rules as illustrated in FIG. 5.

For example, the condition of the practice rule R1 illustrated in FIG. 5 is expressed as follows. Furthermore, information regarding medical care of the practice rule R1 is “M1 medication is recommended”.

( [symbol] disease name. respiratory failure) and ( [symbol] arterial partial pressure of oxygen. numerical value [65>=])

Furthermore, the condition of the practice rule R2 illustrated in FIG. 5 is expressed as follows. Furthermore, information regarding medical care of the practice rule R2 is “M2 medication is recommended”.

( [symbol] disease name. heart failure [symbol] chronic heart failure) and ( [symbol] blood pressure. numerical value [200<=])

Furthermore, a condition of a practice rule R3 illustrated in FIG. 5 is expressed as follows. Furthermore, information regarding medical care of the practice rule R3 is “M3 medication is recommended”.

( [symbol] disease name. heart failure [symbol] chronic heart failure) and ( [symbol] blood pressure. numerical value [200<=]) and ( [symbol] urine protein test. positive)

The inference processing unit 124 makes the disease name and the practice information input information and determines whether the input information corresponds to the disease name and the practice information of any one of a plurality of practice rules. Note that the inference processing unit 124 refers to the disease name ontology stored in the disease name dictionary storage unit 120, determines whether the input disease name and the disease name in the practice rule are in an inclusion relationship, and then determines whether the input information corresponds to the practice rule. Note that the expression that “the input information corresponds to the disease name and the practice information of the practice rule” is hereinafter also simply expressed as “corresponds to the practice rule”.

For example, a case is considered where input information P_{α} indicating a disease name and practice information of a patient α and input information P_{β} indicating a disease name and practice information of a patient β are given as follows.

P_{α}: ( [symbol] disease name. acute cerebral infarction) and ( [symbol] SBP value. {250}) P_{β}: ( [symbol] disease name. lacunar infarction) and ( [symbol] SBP value. {190}))

In this case, the disease name of the patient α is “acute cerebral infarction” and the SBP value is “250”. Furthermore, the disease name of the patient β is “lacunar infarction” and the SBP value is “190”.

The input information P_{α} of the patient α and the input information P_{β} of the patient β are applied to a following condition of a practice rule R.

( [symbol] disease name. cerebral infarction) and ( [symbol] SBP value. numerical value [200<=])

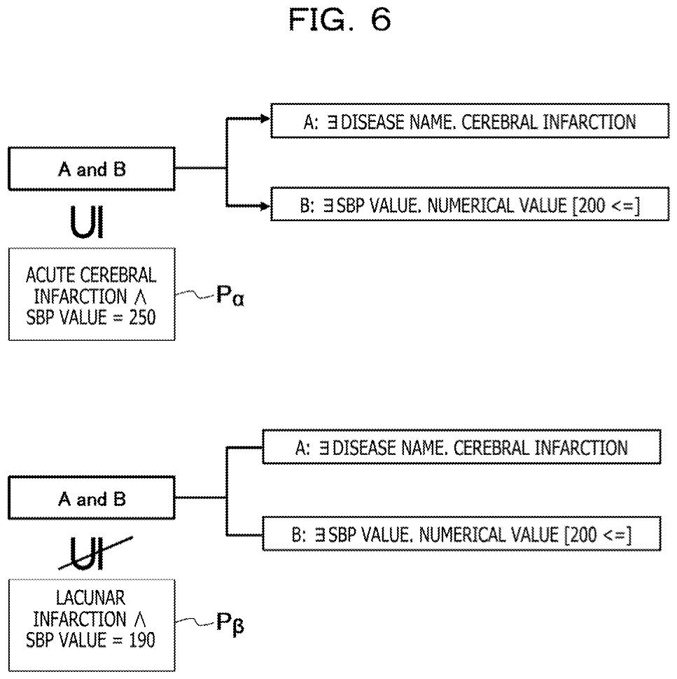

FIG. 6 is an illustration diagram in a case where the input information P_{α} of the patient α and the input information P_{β} of the patient β are applied to the condition of the practice rule R. As illustrated in FIG. 6, the condition of the practice rule R is indicated by a condition A regarding the disease name: ( [symbol] disease name. cerebral infarction) and a condition B regarding the practice information: ( [symbol] SBP value. numerical value [200<=]) (represented with “and” in FIG. 6).

In this case, as illustrated in FIG. 6, since the disease name of the patient α is “acute cerebral infarction”, this does not match the condition A regarding the disease name “cerebral infarction” In the practice rule R. However, the inference processing unit 124 refers to the disease name ontology, determines whether the input disease name “acute cerebral infarction” and the disease name “cerebral infarction” in the practice rule R are in the inclusion relationship, and determines whether this corresponds to the practice rule R.

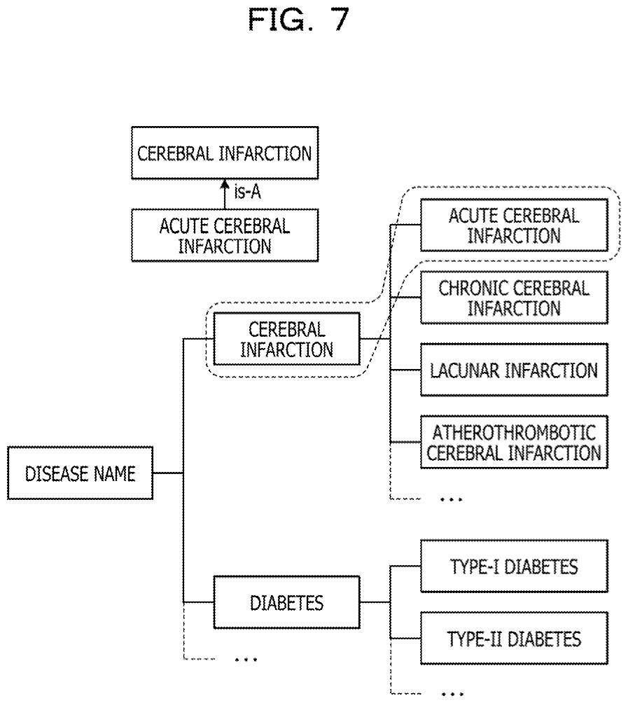

Specifically, the inference processing unit 124 refers to the disease name ontology, and determines that the disease name “acute cerebral infarction” is the subordinate concept of the disease name “cerebral infarction” and they are in an “is-A” relationship in the ontology as illustrated in FIG. 7. Therefore, the inference processing unit 124 determines that the disease name “acute cerebral infarction” of the patient α and the disease name “cerebral infarction” of the condition A regarding the disease name in the practice rule R are in the inclusion relationship.

Furthermore, the inference processing unit 124 determines whether the practice information “SBP value=250” of the patient corresponds to ( [symbol] SBP value. numerical value [200<=]) in the practice rule R. Since the SBP value of the patient α is 250, this satisfies the condition B regarding the practice information that the “SBP value” is 200 or higher. Therefore, the inference processing unit 124 determines that the input information P_{α} of the patient α corresponds to the practice rule R.

Note that, in this case, since the input information P_{α} of the patient α is a subset of a set that satisfies the condition A and the condition B, a following relational expression is established.

P_{α} [symbol] A and B

On the other hand, since the disease name of the patient β is “lacunar infarction”, this does not match the condition A “cerebral infarction” of the practice rule R. However, the inference processing unit 124 refers to the disease name ontology and determines that the disease name “lacunar infarction” of the patient β and the disease name “cerebral infarction” of the condition A regarding the disease name in the practice rule R are in the inclusion relationship as illustrated in FIG. 7.

Furthermore, the inference processing unit 124 determines whether the practice information “SBP value=190” of the patient corresponds to ( [symbol] SBP value. numerical value [200<=]) in the practice rule R. Since the SBP value of the patient β is 190, this does not satisfy the condition B regarding the practice information that the “SBP value” is 200 or higher. Therefore, the inference processing unit 124 determines that the input information P_{β} of the patient β does not correspond to the practice rule R.

Note that, in this case, regarding the input information P_{β} of the patient β, a following relational expression is established between the condition A and the condition B.

P_{β} [symbol] A and B

As described above, the inference processing unit 124 determines whether the input disease name and practice information correspond to any one of a plurality of practice rules.

In a case where the input target disease name is not registered in the disease name dictionary, the information presentation device 14 outputs a disease name medically synonymous with or similar to the target disease name as a candidate disease name. As illustrated in FIG. 2, the information presentation device 14 is provided with an acquisition unit 140, a specification unit 142, a determination unit 144, a relationship table storage unit 146, and a presentation unit 148. The relationship table storage unit 146 is an example of a storage unit of the disclosed technology.

The acquisition unit 140 acquires the target disease name of the patient and the practice information of the patient. The information acquired by the acquisition unit 140 is, for example, information input by a user such as a doctor.

The specification unit 142 determines whether the target disease name acquired by the acquisition unit 140 is registered in the disease name ontology stored in the disease name dictionary storage unit 120. Specifically, the specification unit 142 determines whether the target disease name is registered in the disease name ontology by searching the disease name ontology for the target disease name.

Then, in a case where the target disease name is not registered in the disease name ontology, the specification unit 142 specifies the similar disease name similar to the target disease name from the disease name ontology. There may be a plurality of similar disease names or one similar disease name. In this embodiment, a case where a plurality of similar disease names is specified is described as an example.

Note that the similar disease name is the disease name in which a character string satisfies a predetermined criterion with respect to a character string representing the target disease name. For example, the specification unit 142 makes a disease name in which a predetermined number of final characters coincide with those of the target disease name the similar disease name. In this case, the specification unit 142 specifies disease names “acute heart failure”, “chronic heart failure”, “heart failure”, “myocardial failure” and the like in which two final characters “failure” coincide with those of the target disease name “diastolic failure” as the similar disease names.

Alternatively, for example, the specification unit 142 makes a disease name in which a predetermined number of leading characters coincide with those of the target disease name the similar disease name. In this case, disease names “heart failure”, “myocardial failure” and the like in which one leading character “heart” coincides with that of the target disease name “diastolic failure” are specified as the similar disease names.

Alternatively, for example, the specification unit 142 makes a disease name having a predetermined number or more of common characters with the target disease name the similar disease name. In this case, the disease names “heart failure”, “myocardial failure” and the like in which two characters “failure” coincide with those of the target disease name “diastolic failure” are specified as the similar disease names.

The determination unit 144 allows the inference device 12 to determine whether each of a plurality of similar disease names specified by the specification unit 142 corresponds to any one of a plurality of practice rules.

Specifically, for each of a plurality of similar disease names, the determination unit 144 inputs the input information that is the combination of the similar disease name and the practice information to the inference processing unit 124 of the inference device 12.

The inference processing unit 124 of the inference device 12 refers to the disease name ontology stored in the disease name dictionary storage unit 120 and, for each of a plurality of similar disease names, determines whether the combination of the similar disease name and the practice information corresponds to any one of a plurality of practice rules in the practice rule storage unit 122. Then, the inference processing unit 124 of the inference device 12 outputs the similar disease name that corresponds to at least one of a plurality of practice rules and the practice rule to which the similar disease name corresponds. Note that the inference processing unit 124 of the inference device 12 outputs a determination result also in a case where the similar disease name corresponds to none of a plurality of practice rules. Note that the practice rule to which the similar disease name corresponds may be plural or one according to cases.

Then, the determination unit 144 acquires the determination result by the inference device 12. Specifically, the determination unit 144 acquires the similar disease name that corresponds to any one of a plurality of practice rules and the practice rule to which the similar disease name corresponds. Therefore, even in a case where the target disease name is not registered in the disease name dictionary, the disease name that is medically similar to the target disease name is appropriately searched for. Furthermore, even in a case where the similar disease name corresponds to none of a plurality of practice rules, the determination unit 144 acquires the determination result. Then, the determination unit 144 stores the determination result by the inference device 12 in the relationship table storage unit 146.

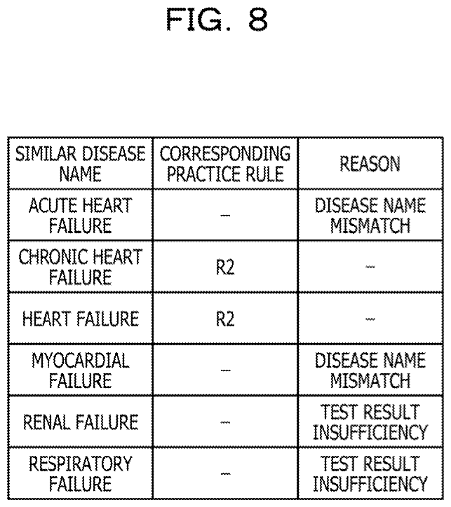

A table as illustrated in FIG. 8 is stored, for example, in the relationship table storage unit 146. In the table illustrated in FIG. 8, the similar disease name specified by the specification unit 142, the practice rule to which this corresponds, or a reason in a case where the similar disease name does not correspond to the practice rule are stored in association with each other. In the table illustrated in FIG. 8, it is illustrated that the similar disease name “chronic heart failure” and the similar disease name “heart failure” correspond to the practice rule R2. Furthermore, in the table illustrated in FIG. 8, it is illustrated that the similar disease name “acute heart failure”, the similar disease name “myocardial failure”, the similar disease name “renal failure”, and the similar disease name “respiratory failure” correspond to none of the practice rules.

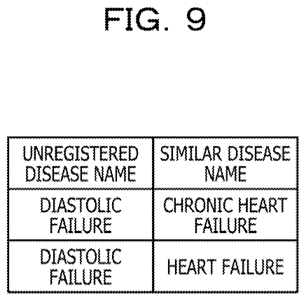

Furthermore, the relationship table storage unit 146 further stores a relationship table in which the target disease name that is not registered in the disease name ontology and the similar disease name that corresponds to the practice rule are associated with each other. An unregistered disease name corresponding to the target disease name and the similar disease name that corresponds to the practice rule are stored in a table form, for example, as illustrated in FIG. 9. In the table illustrated in FIG. 9, the unregistered disease name and the similar disease name are stored in association with each other. In the example illustrated in FIG. 9, the target disease name “diastolic failure” that is the unregistered disease name and the similar disease name “chronic heart failure” that corresponds to the practice rule are stored in association with each other. Furthermore, in the example illustrated in FIG. 9, the target disease name “diastolic failure” that is the unregistered disease name and the similar disease name “heart failure” that corresponds to the practice rule are stored in association with each other.

The presentation unit 148 presents the similar disease name determined to correspond to any one of a plurality of practice rules as the candidate disease name. Specifically, the presentation unit 148 presents the candidate disease name that is the similar disease name determined to correspond to any one of a plurality of practice rules and the practice rule to which the candidate disease name that is the similar disease name corresponds stored in the relationship table storage unit 146. Note that the presentation unit 148 may also present only the similar disease name determined to correspond to the practice rule without presenting the practice rule.

The medical worker such as the doctor who is the user confirms the candidate disease name and the practice rule presented by the presentation unit 148 and confirms whether the target disease name input by himself/herself corresponds to the candidate disease name.

Note that, here, in a case where there is a plurality of candidate disease names determined to correspond to the practice rule, the user specifies the disease name that is considered to be most suitable for the target disease name from the plurality of candidate disease names and inputs the same to the information presentation device 14.

The acquisition unit 140 of the information presentation device 14 acquires the candidate disease name input by the user as a specified disease name. Then, the acquisition unit 140 updates contents of the relationship table storage unit 146 on the basis of the specified disease name. Specifically, the acquisition unit 140 stores the target disease name in the relationship table storage unit 146 in association with the specified disease name.

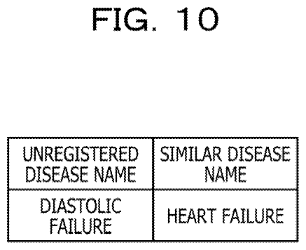

For example, in a case where “chronic heart failure” and “heart failure” are presented to the user as the candidate disease names and then the user selects “heart failure”, the acquisition unit 140 updates the contents of the relationship table storage unit 146 so that a state illustrated in FIG. 10 is realized. The table stored in the relationship table storage unit 146 is referred to when the target disease name is input from next time onward.

From next time onward, the determination unit 144 executes processing with reference to the information stored in the relationship table storage unit 146. Specifically, in a case where the determination unit 144 receives the input of the target disease name associated with the specified disease name, this replaces the target disease name with the specified disease name and makes the specified disease name and the practice information corresponding to the target disease name the input information. Then, the determination unit 144 inputs a combination of the specified disease name and the practice information to the inference processing unit 124 of the inference device 12 and obtains the practice rule to which the specified disease name corresponds. For example, from next time onward, in a case where “diastolic failure” is input as the target disease name, the determination unit 144 refers to the table stored in the relationship table storage unit 146 and executes the processing supposing that “diastolic failure” is “heart failure”.

Then, the presentation unit 148 presents the specified disease name and the practice rule to which the specified disease name corresponds. Therefore, the user acquires the information regarding medical care (for example, information of medication and the like) included in the practice rule.

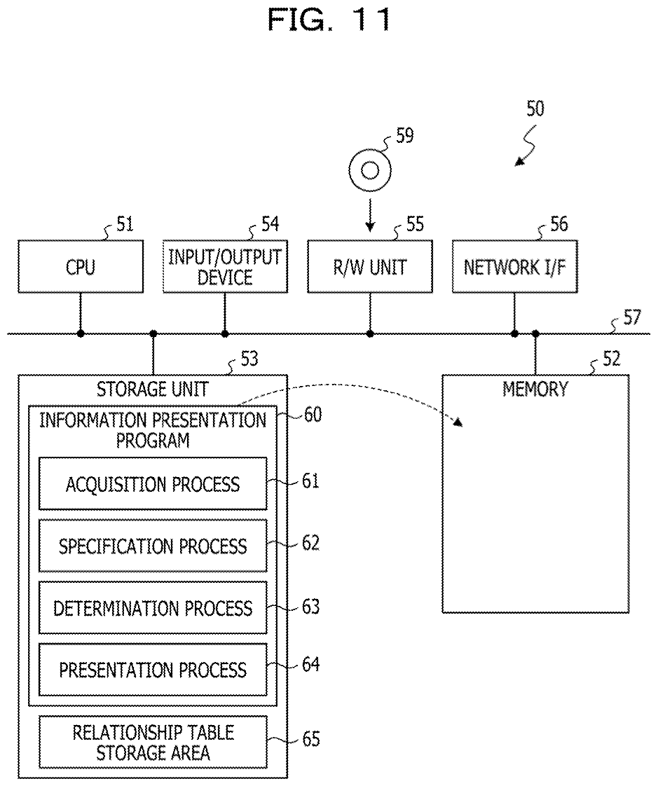

The information presentation device 14 may be realized, for example, by a computer 50 illustrated in FIG. 11. The computer 50 is provided with a central processing unit (CPU) 51, a memory 52 as a temporary storage area, and a nonvolatile storage unit 53. Furthermore, the computer 50 is provided with an input/output interface (I/F) 54 to which an input/output device (display device and the like) is connected, and a read/write (R/W) unit 55 that controls reading and writing of data from and to a recording medium 59. Furthermore, the computer 50 is provided with a network I/F 56 connected to a network such as the Internet. The CPU 51, the memory 52, the storage unit 53, the input/output I/F 54, the R/W unit 55, and the network I/F 56 are connected to each other via a bus 57.

The storage unit 53 may be realized by a hard disk drive (HDD), a solid state drive (SSD), a flash memory and the like. The storage unit 53 as a storage medium stores an information presentation program 60 for allowing the computer 50 to serve as the information presentation device 14. The information presentation program 60 includes an acquisition process 61, a specification process 62, a determination process 63, and a presentation process 64. The information described in the description regarding the relationship table storage unit 146 is stored in a relationship table storage area 65.

The CPU 51 reads out the information presentation program 60 from the storage unit 53, expands the same in the memory 52, and sequentially executes the processes included in the information presentation program 60. The CPU 51 executes the acquisition process 61, thereby operating as the acquisition unit 140 illustrated in FIG. 2. Furthermore, the CPU 51 executes the specification process 62, thereby operating as the specification unit 142 illustrated in FIG. 2. Furthermore, the CPU 51 executes the determination process 63, thereby operating as the determination unit 144 illustrated in FIG. 2. Furthermore, the CPU 51 executes the presentation process 64, thereby operating as the presentation unit 148 illustrated in FIG. 2. Furthermore, the CPU 51 reads out the information from the relationship table storage area 65, thereby expanding the relationship table storage unit 146 in the memory 52. Therefore, the computer 50 that executes the information presentation program 60 serves as the information presentation device 14. The CPU 51 that executes the information presentation program 60 as software is hardware.

Note that the functions realized by the information presentation program 60 may also be realized by, for example, a semiconductor integrated circuit, in further detail, an application specific integrated circuit (ASIC) and the like.

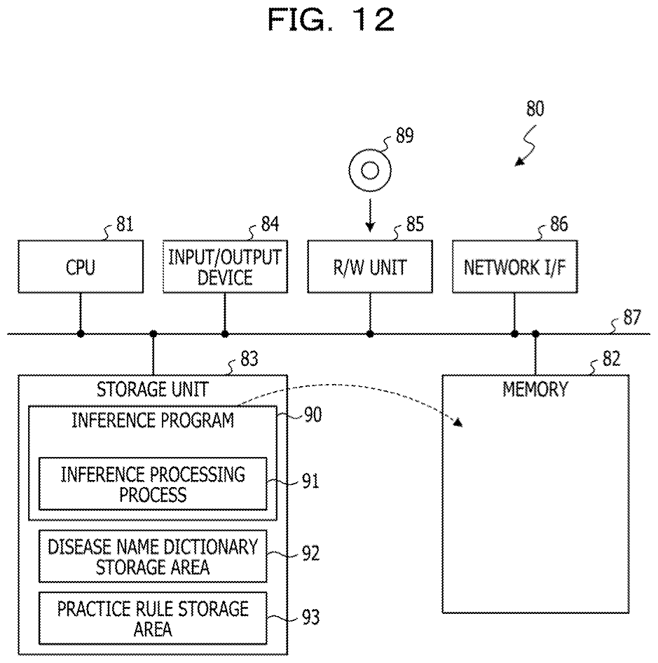

The inference device 12 may be realized by, for example, a computer 80 illustrated in FIG. 12. The computer 80 is provided with a CPU 81, a memory 82 as a temporary storage area, and a nonvolatile storage unit 83. Furthermore, the computer 80 is provided with an input/output I/F 84 to which an input/output device is connected, and an R/W unit 85 that controls reading and writing of data from and to a recording medium 89. Furthermore, the computer 80 is provided with a network I/F 86 connected to a network such as the Internet. The CPU 81, the memory 82, the storage unit 83, the input/output I/F 84, the R/W unit 85, and the network I/F 86 are connected to each other via a bus 87.

The storage unit 83 may be realized by a HDD, an SSD, a flash memory and the like. The storage unit 83 as a storage medium stores an inference program 90 for allowing the computer 80 to serve as the inference device 12. The inference program 90 includes an inference processing process 91. A disease name dictionary storage area 92 stores the information described in the description regarding the disease name dictionary storage unit 120. The information described in the description regarding the practice rule storage unit 122 is stored in a practice rule storage area 93.

The CPU 81 reads out the inference program 90 from the storage unit 83, expands the same in the memory 82, and sequentially executes the processes included in the inference program 90. The CPU 81 executes the inference processing process 91, thereby operating as the inference processing unit 124 illustrated in FIG. 2. Furthermore, the CPU 81 reads out the information from the disease name dictionary storage area 92, and expands the disease name dictionary storage unit 120 in the memory 82. Furthermore, the CPU 81 reads out the information from the practice rule storage area 93, and expands the practice rule storage unit 122 in the memory 82. Therefore, the computer 80 that executes the inference program 90 serves as the inference device 12. The CPU 81 that executes the inference program 90 as software is hardware.

Note that the function realized by the inference program 90 may also be realized by, for example, a semiconductor integrated circuit, more specifically, an ASIC and the like.

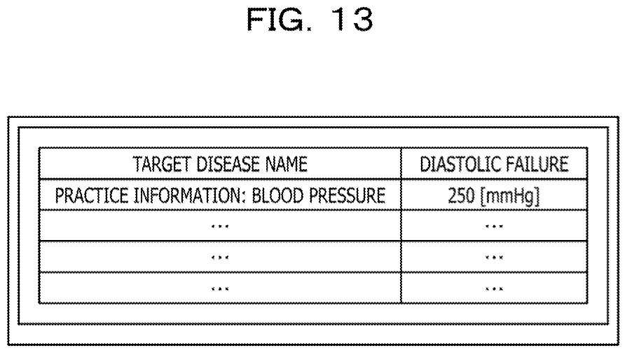

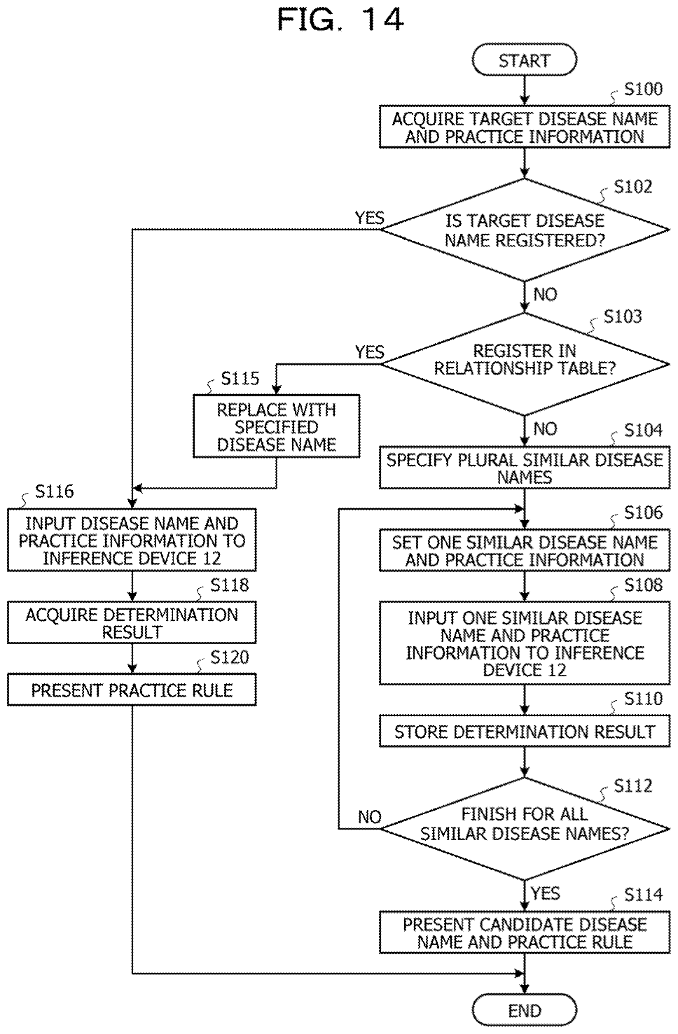

Next, an action of the information presentation device 14 and the inference device 12 according to this embodiment is described. First, the user inputs the target disease name and the practice information to the information presentation device 14. For example, a display device (not illustrated) of the information presentation device 14 displays a screen as illustrated in FIG. 13. Therefore, the user inputs the target disease name “diastolic failure” and the practice information “blood pressure=250” to the information presentation device 14. When the information is input, the information presentation device 14 executes an information presentation routine illustrated in FIG. 14.

At step S100, the acquisition unit 140 acquires the target disease name and the practice information input by the user. For example, the acquisition unit 140 acquires the target disease name “diastolic failure” and the practice information “blood pressure=250”.

At step S102, the specification unit 142 determines whether the target disease name is registered in the disease name ontology stored in the disease name dictionary storage unit 120 of the inference device 12. In a case where the target disease name is registered in the disease name ontology, the procedure shifts to step S116. On the other hand, in a case where the target disease name is not registered in the disease name ontology, the procedure shifts to step S103.

At step S103, the determination unit 144 determines whether the target disease name acquired at step S100 described above is registered in the relationship table stored in the relationship table storage unit 146. In a case where the target disease name is registered in the relationship table, the procedure shifts to step S115. On the other hand, in a case where the target disease name is not registered in the relationship table, the procedure shifts to step S104.

At step S104, the specification unit 142 specifies a plurality of similar disease names similar to the target disease name from the disease name ontology stored in the disease name dictionary storage unit 120. Specifically, the specification unit 142 specifies a plurality of similar disease names the degree of similarity in character string with the target disease name of which satisfies a predetermined criterion. For example, the specification unit 142 specifies the similar disease names “acute heart failure”, “chronic heart failure”, “heart failure”, “myocardial failure”, “renal failure”, and “respiratory failure” in which final two characters coincide with those of the target disease name “diastolic failure”.

At step S106, the determination unit 144 sets any one of a plurality of similar disease names specified at step S104 described above and the practice information acquired at step S100 described above as the input information.

At step S108, the determination unit 144 inputs the input information set at step S106 described above to the inference device 12.

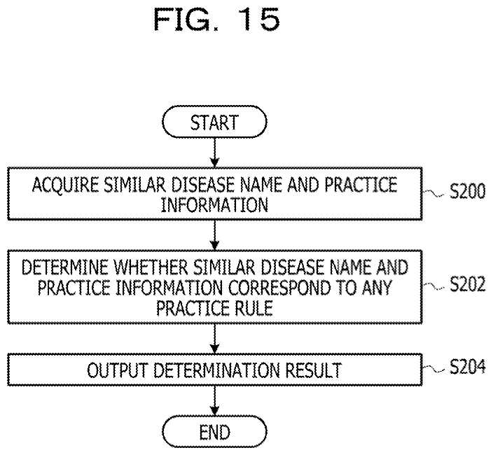

Upon receiving the similar disease name and the practice information output from the information presentation device 14, the inference device 12 executes an inference routine illustrated in FIG. 15.

At step S200, the inference processing unit 124 of the inference device 12 acquires the input information that is the combination of the similar disease name and the practice information output from the information presentation device 14.

At step S202, the inference processing unit 124 determines whether the input information acquired at step S200 described above corresponds to the disease name and the practice information of any one of a plurality of practice rules stored in the practice rule storage unit 122. Note that the inference processing unit 124 refers to the disease name ontology stored in the disease name dictionary storage unit 120 and determines whether the similar disease name and the disease name in the practice rule are in an inclusion relationship, thereby determining whether the input information corresponds to any one of the practice rules.

At step S204, the inference processing unit 124 outputs the determination result acquired at step S202 described above to the information presentation device 14.

Returning to FIG. 14, at step S110, the determination unit 144 stores the determination result output from the inference device 12 at step S204 described above in the relationship table storage unit 146.

At step S112, the determination unit 144 determines whether the processes at steps S106 to S110 described above are executed for all the similar disease names specified at step S104 described above. In a case where the processes at steps S106 to S110 described above are executed for all the similar disease names specified at step S104 described above, the procedure shifts to step S114. In contrast, in a case where there is the similar disease name for which the processes at steps S106 to S110 described above are not executed, the procedure returns to step S106.

By repeating the processes at steps S106 to S110 described above, it is determined whether each of a plurality of similar disease names corresponds to the practice rule.

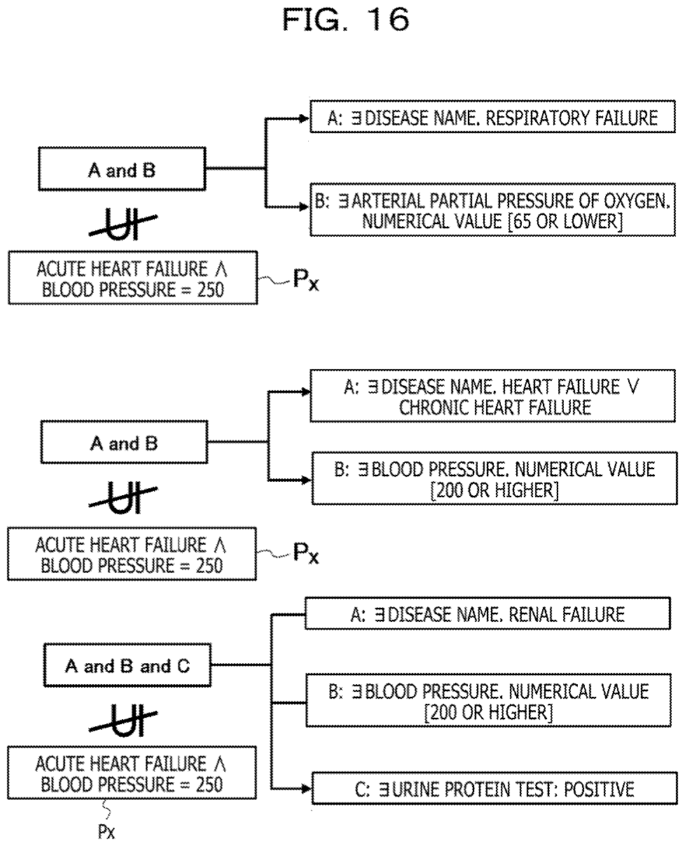

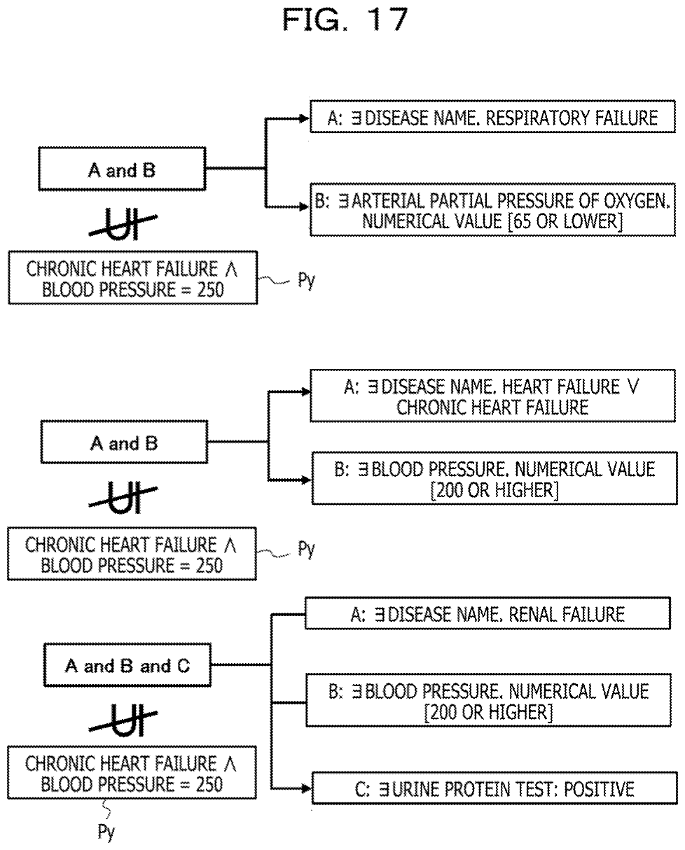

Therefore, for example, as illustrated in FIG. 16, a determination result that input information Px that is a combination of the similar disease name “acute heart failure” and the practice information “blood pressure=250”, does not correspond to all the practice rules including the practice rule R1, the practice rule R2, and the practice rule R3 is obtained. Furthermore, as illustrated in FIG. 17, a determination result that input information Py that is a combination of the similar disease name “chronic heart failure” and the practice information “blood pressure=250” corresponds to the condition of the practice rule R2 out of the practice rule R1, the practice rule R2, and the practice rule R3 is obtained.

In this case, the inference processing unit 124 obtains as a determination result that the similar disease name “acute heart failure” does not correspond to any practice rule and the similar disease name “chronic heart failure” corresponds to the practice rule R2.

At step S114, the presentation unit 148 presents the similar disease name determined to correspond to any one of a plurality of practice rules as a candidate disease name of the target disease name acquired at step S100 described above on the basis of the determination result stored in the relationship table storage unit 146. Furthermore, when presenting the candidate disease name, the presentation unit 148 presents the practice rule to which the candidate disease name corresponds.

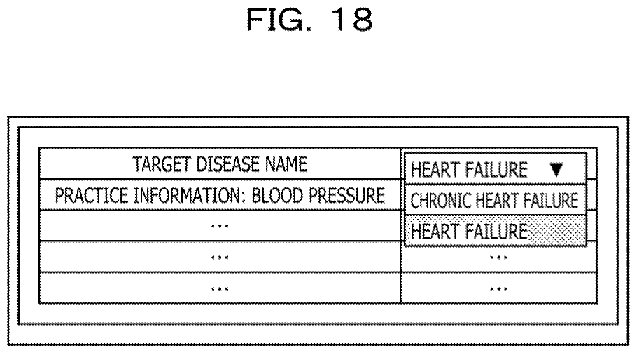

The display device (not illustrated) acquires the information output from the presentation unit 148. Then, the display device (not illustrated) displays, for example, a screen as illustrated in FIG. 18.

On the screen illustrated in FIG. 18, “heart failure” and “chronic heart failure” are displayed as the candidate disease names. The user confirms the candidate disease names and selects the candidate disease name corresponding to the target disease name “diastolic failure”.

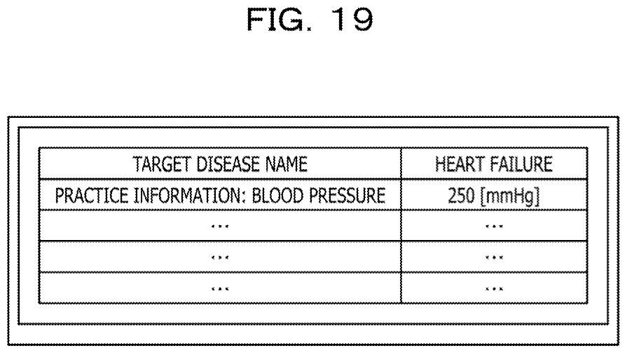

For example, when the user selects the candidate disease name “heart failure” as the disease name corresponding to “diastolic failure”, a target disease name field is changed as in a screen illustrated in FIG. 19.

In this case, the acquisition unit 140 of the information presentation device 14 stores the disease name “heart failure” selected by the user in the relationship table storage unit 146 in association with the target disease name “diastolic failure”. Therefore, for example, a table as illustrated in FIG. 10 is stored in the relationship table storage unit 146.

From next time onward, in a case where the target disease name is input, the determination unit 144 of the information presentation device 14 refers to the relationship table stored in the relationship table storage unit 146 and specifies the disease name corresponding to the target disease name.

Note that the processes at steps S115 to S120 are the processes in a case where the target disease name is registered in the disease name ontology, or in a case where the target disease name is registered in the relationship table.

At step S115, the determination unit 144 refers to the relationship table stored in the relationship table storage unit 146, and replaces the target disease name acquired at step S100 described above with the corresponding specified disease name.

At step S116, the determination unit 144 makes a combination of the target disease name acquired at step S100 described above or the specified disease name replaced at step S115 described above and the practice information acquired at step S100 described above input information, and inputs the same to the inference device 12.

Then, upon receiving the input information output from the information presentation device 14, the inference device 12 executes the inference routine illustrated in FIG. 15.

At step S118, the determination unit 144 acquires the practice rule output from the inference device 12 at step S116 described above.

At step S120, the presentation unit 148 presents the practice rule acquired at step S118 described above and the target disease name acquired at step S100 described above or the specified disease name replaced at step S115 described above.

The user confirms the practice rule corresponding to the target disease name input by himself/herself or the specified disease name with which the target disease name is replaced. Then, the user confirms the information regarding medical care in the practice rule.

As described above, in a case where the target disease name is not registered in the disease name dictionary, the information presentation device according to this embodiment specifies at least one similar disease name similar to the target disease name from the disease name dictionary because the character string of each of them satisfies a predetermined criterion with the character string representing the target disease name. Furthermore, the information presentation device makes the specified similar disease name and the practice information corresponding to the target disease name the input information, and determines whether this corresponds to the disease name and the practice information of any one of a plurality of practice rules by using the inference device. The inference device makes the disease name and the practice information the input information, and determines whether the input information corresponds to the disease name and the practice information of any one of a plurality of practice rules in each of which the relationship between the disease name, the practice information, and the information regarding medical care is determined. The information presentation device presents the similar disease name determined to correspond to the disease name and the practice information of any one of a plurality of practice rules as the candidate disease name of the target disease name. Therefore, even in a case where the input disease name is not registered in the disease name dictionary, the disease name medically similar to the input disease name may be appropriately searched for. Furthermore, by presenting only the similar disease names corresponding to the practice rule as the candidate disease names, the information presentation device of this embodiment presents may efficiently obtain the disease name medically similar to the target disease name as compared to a case where the disease names similar in character string are presented.

Furthermore, the disease name dictionary of this embodiment is the disease name ontology in which a plurality of disease names is registered so as to indicate the inclusion relationship between the disease names. Therefore, the information presentation device according to this embodiment determines whether the similar disease name corresponds to the practice rule depending on whether the similar disease name and the disease name in the practice rule are in the inclusion relationship. Therefore, even in a case where the similar disease name and the disease name in the practice rule are written in different manners, the correspondence relationship between the disease names is appropriately determined, and it may be appropriately determined whether the similar disease name corresponds to the practice rule.

Furthermore, the information presentation device according to this embodiment further presents the practice rule to which the candidate disease name corresponds when presenting the candidate disease name. Therefore, a doctor and the like being a user may confirm whether the candidate disease name is the disease name medically similar to the target disease name in consideration of the practice rule, and appropriately specify the disease name medically similar to the target disease name.

The information presentation device according to this embodiment associates the target disease name with the specified disease name specified from the candidate disease names and records them in the relationship table. Therefore, in subsequent determination regarding correspondence of the practice rule, the correspondence relationship registered in the relationship table is referred to, the target disease name that is not registered in the disease name dictionary is replaced with the corresponding disease name, and it is determined whether this corresponds to the practice rule. Therefore, it is determined whether this corresponds to the practice rule more efficiently.

### Example 1

Next, an example is described. Using the information presentation device of this embodiment, an experiment was conducted to search for a disease name medically similar to a target disease name “diastolic failure”. Furthermore, as a comparative example, an experiment was conducted to search for a disease name medically similar to a target disease name “diastolic failure” by using only a degree of similarity in character string, which is the conventional technology.

In the comparative example, 173 similar disease names “* failure” similar to “diastolic failure” were specified. Note that “*” represents an arbitrary character. Then, on the basis of judgment of a doctor, since “diastolic failure” is a disease name medically corresponding to “heart failure”, among 173 specified similar disease names, 13 disease names written as “* heart failure” were selected by the doctor. The doctor came to make a judgment about the 13 cases.

In contrast, in the example, 173 similar disease names “* failure” similar to “diastolic failure” were specified, and two disease names corresponding to a practice rule were presented as candidate disease names. Therefore, the doctor only had to make judgment about two disease names.

Therefore, it is understood that the disease name medically similar to the target disease name may be efficiently obtained according to this embodiment as compared with a case of searching for a disease name similar in character string.

Note that although a mode in which each program is stored (installed) in advance in a storage unit is described above, there is no limitation. The program regarding the disclosed technology may also be provided in the form recorded on a recording medium such as a compact disc read only memory (CD-ROM), a digital versatile disc read only memory (DVD-ROM), and a universal serial bus (USB) memory.

All the documents, patent applications, and technical standards described in this specification are herein incorporated by reference in the same degree as in a case where each of documents, patent applications, and technical standards is specifically and individually indicated to be incorporated by reference.

Next, a variation of this embodiment is described.

The information presentation device of the above-described embodiment is applicable even when a target disease name is written in an old style. For example, in a case where “sugar” in “glycosuria” is written in the old style, “glycosuria”, “renal glycosuria”, and “proteinuria” are specified as similar disease names, and it is determined whether each of the similar disease names corresponds to a practice rule. Then, the similar disease name determined to correspond to the practice rule is presented as a candidate disease name of the target disease name in which “sugar” in “glycosuria” is written in the old style.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable storage medium for storing a program which causes a processor to perform processing, the processing comprising:
in response to inputting of a target disease name and an input practice information with respect to the target disease, detecting a similar disease name from a list of disease names, the similar disease name being a disease name having a character string similar to a character string representing the target disease name by a predetermined criterion, the input practice information indicating an examination result when the target disease name is determined in a practice;
obtaining a determination result by inputting input information to an inference task, the input information including the detected similar disease name and the input practice information, the inference task being configured to determine whether the similar disease name corresponds to any one of a plurality of practice rules, each of the plurality of practice rules defining a relationship between a corresponding disease name and a practice information with respect to the corresponding disease name; and
in response to the determination result indicating that the similar disease name corresponds to any one of the plurality of practice rules, displaying the similar disease name.

2. The non-transitory computer-readable storage medium according to claim 1,
wherein, when determining whether the input information corresponds to the disease name and the practice information of any one of the plurality of practice rules,
the inference task refers to disease name ontology in which a plurality of disease names is registered so as to indicate an inclusion relationship between disease names, determines whether the similar disease name and the disease name in the practice rule are in the inclusion relationship, and determines whether the input information corresponds to the disease name and the practice information of the practice rule.

3. The non-transitory computer-readable storage medium according to claim 1,
wherein, when the similar disease name is presented, the practice rule to which the similar disease name corresponds is further presented.

4. The non-transitory computer-readable storage medium according to claim 1,
wherein, the target disease name is stored in a storage unit in association with a specified disease name specified from the similar disease name,
in a case where an input of the target disease name associated with the specified disease name is received, the target disease name is replaced with the specified disease name, the specified disease name and practice information corresponding to the target disease name is made the input information, and it is determined whether the input information corresponds to any one of the plurality of practice rules; and
the specified disease name and the practice rule to which the specified disease name corresponds are presented.

5. The non-transitory computer-readable storage medium according to claim 2,
wherein, in a case where a plurality of similar disease names is specified when the similar disease name similar to the target disease name is specified from the disease name ontology, it is determined whether each of a specified plurality of similar disease names corresponds to any one of the plurality of practice rules.

6. An information presentation device comprising:
a memory; and
a processor coupled to the memory, the processor being configured to perform processing, the processing including:
in response to inputting of a target disease name and an input practice information with respect to the target disease, detecting a similar disease name from a list of disease names, the similar disease name being a disease name having a character string similar to a character string representing the target disease name by a predetermined criterion, the input practice information indicating an examination result when the target disease name is determined in a practice;
obtaining a determination result by inputting input information to an inference task, the input information including the detected similar disease name and the input practice information, the inference task being configured to determine whether the similar disease name corresponds to any one of a plurality of practice rules, each of the plurality of practice rules defining a relationship between a corresponding disease name and a practice information with respect to the corresponding disease name; and
in response to the determination result indicating that the similar disease name corresponds to any one of the plurality of practice rules, displaying the similar disease name.

7. The information presentation device according to claim 6,
wherein, when determining whether the input information corresponds to the disease name and the practice information of any one of the plurality of practice rules,
the inference task refers to disease name ontology in which a plurality of disease names is registered so as to indicate an inclusion relationship between disease names, determines whether the similar disease name and the disease name in the practice rule are in the inclusion relationship, and determines whether the input information corresponds to the disease name and the practice information of the practice rule.

8. The information presentation device according to claim 6,
wherein, when the similar disease name is presented, the practice rule to which the similar disease name corresponds is further presented.

9. The information presentation device according to claim 6,
wherein, the target disease name is stored in a storage unit in association with a specified disease name specified from the similar disease name,
in a case where an input of the target disease name associated with the specified disease name is received, the target disease name is replaced with the specified disease name, the specified disease name and practice information corresponding to the target disease name is made the input information, and it is determined whether the input information corresponds to any one of the plurality of practice rules; and
the specified disease name and the practice rule to which the specified disease name corresponds are presented.

10. The information presentation device according to claim 7,
wherein, in a case where a plurality of similar disease names is specified when the similar disease name similar to the target disease name is specified from the disease name ontology, it is determined whether each of a specified plurality of similar disease names corresponds to any one of the plurality of practice rules.

11. An information presentation method implemented by a computer, the method comprising:
in response to inputting of a target disease name and an input practice information with respect to the target disease, detecting a similar disease name from a list of disease names, the similar disease name being a disease name having a character string similar to a character string representing the target disease name by a predetermined criterion, the input practice information indicating an examination result when the target disease name is determined in a practice;
obtaining a determination result by inputting input information to an inference task, the input information including the detected similar disease name and the input practice information, the inference task being configured to determine whether the similar disease name corresponds to any one of a plurality of practice rules, each of the plurality of practice rules defining a relationship between a corresponding disease name and a practice information with respect to the corresponding disease name; and
in response to the determination result indicating that the similar disease name corresponds to any one of the plurality of practice rules, displaying the similar disease name.

12. The information presentation method according to claim 11,
wherein, when determining whether the input information corresponds to the disease name and the practice information of any one of the plurality of practice rules,
the inference task refers to disease name ontology in which a plurality of disease names is registered so as to indicate an inclusion relationship between disease names, determines whether the similar disease name and the disease name in the practice rule are in the inclusion relationship, and determines whether the input information corresponds to the disease name and the practice information of the practice rule.

13. The information presentation method according to claim 11,
wherein, when the similar disease name is presented, the practice rule to which the similar disease name corresponds is further presented.

14. The information presentation method according to claim 11,
wherein, the target disease name is stored in a storage unit in association with a specified disease name specified from the similar disease name,
in a case where an input of the target disease name associated with the specified disease name is received, the target disease name is replaced with the specified disease name, the specified disease name and practice information corresponding to the target disease name is made the input information, and it is determined whether the input information corresponds to any one of the plurality of practice rules; and
the specified disease name and the practice rule to which the specified disease name corresponds are presented.

15. The information presentation method according to claim 12,
wherein, in a case where a plurality of similar disease names is specified when the similar disease name similar to the target disease name is specified from the disease name ontology, it is determined whether each of a specified plurality of similar disease names corresponds to any one of the plurality of practice rules.
